(19) United States
(12) Patent Application Publication
(10) Pub. No.: US 2025/0137985 A1
(43) Pub. Date: May 1, 2025

(54) ASSESSING RELATIVE PLANT NITROGEN IN A FIELD ENVIRONMENT

(71) Applicant: Pivot Bio, Inc., Berkeley, CA (US)

(72) Inventors: Clayton NEVINS, Ames, IA (US); Lori REESE, Mansfield, OH (US); Karsten TEMME, Sonoma, CA (US); Crockett WALTER, Bradford, TN (US)

(73) Assignee: Pivot Bio, Inc., Berkeley, CA (US)

(21) Appl. No.: 18/833,515

(22) PCT Filed: Jan. 27, 2023
(86) PCT No.: PCT/US2023/011735

§ 371 Date: Jul. 26, 2024

Related U.S. Application Data
(60) Provisional application No. 63/303,694, filed on Jan. 27, 2022.

(51) Int. Cl. G01N 33/00; A01C 21/00; G06N 20/00

(52) U.S. Cl. CPC G01N 33/0098; A01C 21/007; G06N 20/00

(57) ABSTRACT

Methods are provided for assessing the nitrogen status of a plant or multiple plants in a plurality of plants, and optionally comparing the nutrient status to a plant or multiple plants in another plurality of plants (e.g., another plurality of plants in a different field or different region of the same field). The methods find utility, for example, in validating the performance of alternative plant nitrogen treatments such as validating the performance of nitrogen fixing microbes in replacing a defined amount of nitrogen from synthetic nitrogen treatment.

### CROSS-REFERENCE TO RELATED APPLICATIONS

This application claims priority to U.S. Provisional Patent Application Ser. No. 63/303,694, filed Jan. 27, 2022; the entire contents of which are herein incorporated by reference.

### TECHNICAL FIELD

This document relates to agriculture and agricultural practices, and more particularly to determining nutrients in a field, for example determining plant nitrogen status of one or more plants in a field based on the chlorophyll content and biomass of the one or more plants. Plant nitrogen status can be used to confirm performance from agricultural practices, such as nutrient management practices or other crop treatments.

### BACKGROUND

Biological nitrogen fixation (BNF) is a process by which plant-associated microbes are believed to be able to provide nitrogen to host plants. Nitrogen is an important nutrient that influences plant growth. In particular, nitrogen is present in both amino acids and chlorophyll pigments, and a wide variety of biological processes, including plant-based protein synthesis and photosynthesis depend on the availability of nitrogen. When adequate soluble nitrogen is not available in a plant's growth medium, vegetative growth may be retarded and fruit production attenuated.

Typically, fixation of atmospheric nitrogen gas to yield soluble ammonia occurs via naturally occurring microbes. Nitrogenases present in the bacteria catalyze atmospheric nitrogen reduction. Significant research activity is currently directed to engineering improved microbes that enhance reductive conversion of atmospheric nitrogen to ammonia as an alternative to prior practices involving synthetic nitrogen fertilizer. An important aspect of this activity is measurement of nitrogen incorporation in plant tissues in the field to confirm the performance of these new agricultural practices in the field.

### SUMMARY

This document is based on methods and materials for determining plant nitrogen status under field conditions. For example, the methods described herein can determine the plant nitrogen status of one or more plants (e.g., a crop plant, such as a maize plant or variety thereof) under field conditions based on the chlorophyll content and biomass of the one or more plants. As described herein, the chlorophyll content of a plant identified in a field or a first region of a field can be determined using, for example, a chlorophyll meter, and the biomass of the plant can be determined using, for example, a digital scale. The chlorophyll content and the biomass can be normalized for the plant, and the plant nitrogen status of the plant can be determined using the normalized chlorophyll content (CC) and the normalized biomass (PB) of the plant. The nitrogen status can be determined with Formula I: Plant Nitrogen Status (NS)=α(PB)+β(CC), where alpha and beta are scaling factors. The methods described herein can be performed in a field.

Provided here in are methods for determining plant nitrogen status of a plant, the method including (a) determining the chlorophyll content of a plant of a plurality of plants identified in a field or a first region of a field; (b) determining the biomass of the plant; (c) normalizing the determined biomass and the determined chlorophyll content for the plant; and (d) determining plant nitrogen status of the plant using the normalized chlorophyll content (CC) and the normalized biomass (PB) of the plant.

In some cases, chlorophyll content is determined using a chlorophyll meter. In some cases, the biomass is determined using a digital scale.

In some cases, the plant nitrogen status of the plant is determined in step (d) using Formula I: Plant Nitrogen Status (NS)=α(PB)+β(CC). In some cases, α is 0.80. In some cases, β is 0.20.

In some cases, step (a) comprises determining the chlorophyll content of each plant of the plurality of plants, and step (b) comprises determining the biomass of each plant of the plurality of plants. In some cases, step (c) comprises normalizing the determined biomass and the determined chlorophyll content for each plant of the plurality of plants. In some cases, step (d) comprises determining the plant nitrogen status of each plant of the plurality of plants.

In some cases, any of the methods provided herein further comprise determining an average nitrogen status for the plants of the plurality of plants (NS1). In some cases, the plurality of plants comprises at least six plants. In some cases, the plurality of plants comprises at least twelve plants.

Any of the methods described herein further comprise (e) determining the chlorophyll content of each plant of an additional plurality of plants identified in a different field or a second region of the field; (f) determining the biomass of each plant of the additional plurality of plants; (g) normalizing the determined biomass and the determined chlorophyll content for each plant of the additional plurality of plants; (h) determining plant nitrogen status of each plant of the additional plurality of plants using the normalized chlorophyll content (CC) and the normalized biomass (PB) for each plant of the additional plurality of plants; and (i) determining an average nitrogen status for the plants of the additional plurality of plants.

In some cases, chlorophyll content is determined in step (e) using a chlorophyll meter. In some cases, the biomass is determined in step (f) using a digital scale. In some cases, the plant nitrogen status of each plant is determined in step (h) using Formula I: Plant Nitrogen Status (NS)=α(PB)+β(CC). In some cases, α is 0.80. In some cases, β is 0.20.

In any of the methods described herein, the method further comprises determining the relative nitrogen status of the plurality of plants as compared to the additional plurality of plants. In some cases, determining the relative nitrogen status comprises comparing the NS1 to the average nitrogen status of the plants of the additional plurality of plants (NS2). In some cases, the relative nitrogen status is determined using Formula II: Relative Nitrogen Status=(NS1/NS2)−1. In some cases, the method further comprises determining a nitrogen performance index (NPI). In some cases, the NPI is determined using Formula III: NPI=(Relative Nitrogen Status)×100%.

In some cases, the additional plurality of plants comprises at least six plants. In some cases, the additional plurality of plants comprises at least twelve plants. In some cases, the additional plurality of plants is from a different field than the plurality of plants. In some cases, the plurality of plants is from a first region of a field and the additional plurality of plants is from a second region of the field. In some cases, the method further comprises determining plant nitrogen content per acre. In some cases, the plant nitrogen content per acre is determined using the relative nitrogen status and the uptake of nitrogen by growth stage.

In some cases, the plants of the plurality of plants and the plants of the additional plurality of plants are cereal plants. In some cases, the plants of the plurality of plants and the plants of the additional plurality of plants are corn plants. In some cases, the plants of the plurality of plants and the plants of the additional plurality of plants are canola plants. In some cases, the plants of the plurality of plants and the plants of the additional plurality of plants are sorghum plants. In some cases, the plants of the plurality of plants and the plants of the additional plurality of plants are wheat plants. In some cases, the plants of the plurality of plants and the plants of the additional plurality of plants are sunflower plants.

In some cases, the plants of the plurality of plants are provided a different nitrogen treatment than the plants of the additional plurality of plants. In some cases, the different nitrogen treatments comprise treatment with synthetic nitrogen and treatment with nitrogen fixing microbes. In some cases, the different nitrogen treatments comprise treatment with different nitrogen fixing microbes. In some cases, the nitrogen fixing microbes comprise microbes identified in table 1.

Any of the methods described herein can further comprise using the relative nitrogen status to validate or deny a claim for compensation under a performance guarantee program.

Also described herein are one or more machine-readable storage devices storing instructions that are executable by one or more processing devices, and upon such execution cause the one or more processing devices to perform operations comprising: (a) receiving a first input on a chlorophyll content of a plant of a plurality of plants identified in a field or a first region of a field; (b) receiving a second input on a biomass of the plant; (c) normalizing the biomass and the chlorophyll content for the plant; (d) determining plant nitrogen status of the plant using the normalized chlorophyll content (CC) and the normalized biomass (PB) of the plant; and (e) outputting the determined plant nitrogen status.

In some cases, the plant nitrogen status of the plant is determined in step (d) using Formula I: Plant Nitrogen Status (NS)=α(PB)+β(CC). In some cases, α is 0.80. In some cases, β is 0.20.

In some cases, step (a) comprises determining the chlorophyll content of each plant of the plurality of plants, and step (b) comprises determining the biomass of each plant of the plurality of plants. In some cases, step (c) comprises normalizing the determined biomass and the determined chlorophyll content for each plant of the plurality of plants. In some cases, step (d) comprises determining the plant nitrogen status of each plant of the plurality of plants. In some cases, the operations further comprise determining an average nitrogen status for the plants of the plurality of plants (NS1).

In some cases, the operations further comprise: (e) determining the chlorophyll content of each plant of an additional plurality of plants identified in a different field or a second region of the field; (f) determining the biomass of each plant of the additional plurality of plants; (g) normalizing the determined biomass and the determined chlorophyll content for each plant of the additional plurality of plants; (h) determining plant nitrogen status of each plant of the additional plurality of plants using the normalized chlorophyll content (CC) and the normalized biomass (PB) for each plant of the additional plurality of plants; and (i) determining an average nitrogen status for the plants of the additional plurality of plants.

In some cases, the plant nitrogen status of each plant is determined in step (h) using Formula I: Plant Nitrogen Status (NS)=α(PB)+β(CC). In some cases, α is 0.80. In some cases, β is 0.20.

In some cases, the operations further comprise determining the relative nitrogen status of the plurality of plants as compared to the additional plurality of plants. In some cases, determining the relative nitrogen status comprises comparing the NS1 to the average nitrogen status of the plants of the additional plurality of plants (NS2). In some cases, the relative nitrogen status is determined using Formula II: Relative Nitrogen Status=(NS1/NS2)−1. In some cases, the operations further comprise determining a nitrogen performance index (NPI). In some cases, the NPI is determined using Formula III: NPI=(Relative Nitrogen Status)×100%. In some cases, the operations further comprise determining plant nitrogen content per acre. In some cases, the plant nitrogen content per acre is determined using the relative nitrogen status and the uptake of nitrogen by growth stage.

Also provided herein are systems including a chlorophyll meter configured to determine the chlorophyll content of a plant of a plurality of plants identified in a field or a first region of a field; a scale configured to determine the biomass of the plant; one or more processing devices configured to normalize the biomass and the chlorophyll content for the plant; and determine plant nitrogen status of the plant using the normalized chlorophyll content (CC) and the normalized biomass (PB) of the plant.

Also provided herein are computer-implemented methods for determining plant nitrogen status of a plant that include: obtaining, from a chlorophyll sensor, a chlorophyll content of a plant; obtaining, from one or more ranging sensors, a plurality of data points representing a structure of a plant; providing the plurality of data points to a trained machine-learning model, the machine-learning model trained to generate an estimate of one or more parameters related to a biomass of the plant; determining, by one or more processing devices based on an output of the trained machine-learning model generated in response to the machine-learning model receiving the plurality of data points, the biomass of the plant; and determining, by the one or more processing devices, plant nitrogen status of the plant based on the chlorophyll content and the biomass of the plant.

In some embodiments of the computer-implemented methods described herein, determining the biomass of the plant based on the output of the trained-machine-learning model comprises: obtaining, from the trained machine-learning model as the one or more parameters related to the biomass of the plant, a first estimate of volume of the plant and a second estimate of a height of the plant; and determining the biomass of the plant based on the first and second estimates.

In some embodiments of the computer-implemented methods described herein, the one or more ranging sensors include at least one Light Detection and Ranging (LiDAR) sensor.

In some embodiments of the computer-implemented methods described herein, the one or more ranging sensors are disposed on a mobile device. In some embodiments of the computer-implemented methods described herein, the one or more ranging sensors are disposed on an unmanned aerial vehicle (UAV). In some embodiments of any of the computer-implemented methods described herein, the one or more ranging sensors are disposed on a land vehicle.

In some embodiments of any of the computer-implemented methods described herein, the one or more parameters related to a biomass of the plant comprises one or more of: stem diameter, plant volume, plant height, or leaf area index.

Some embodiments of any of the computer-implemented methods described herein further include: generating, based on the determined nitrogen status of the plant, a signal to actuate an agricultural dispensing system to dispense one or more substances that affect the nitrogen status. In some embodiments of any of the computer-implemented methods described herein, the one or more substances comprise at least one of: a fertilizer, a nitrogen stabilizer, a nitrification inhibitor, a urease inhibitor, or a microbe.

Some embodiments of any of the computer-implemented methods described herein further include: storing the determined nitrogen status of the plant in a database, wherein the nitrogen status of the plant is linked within the database to field characterization data associated with the plant. In some embodiments of any of the computer-implemented methods described herein, the field characterization data includes at least one of: precipitation data, temperature data, field boundaries data, soil type data, or fertility plan data.

In some embodiments of any of the computer-implemented methods described herein, the plant is treated with an alternative nitrogen treatment. In some embodiments of any of the computer-implemented methods described herein, the alternative nitrogen treatment comprises a nitrogen fixing microbe. In some embodiments of any of the computer-implemented methods described herein, the nitrogen fixing microbe comprises a microbe selected from Table 1.

Some embodiments of any of the computer-implemented methods described herein further include using the determined plant nitrogen status to validate the provision of nitrogen to the plant by the alternative nitrogen treatment.

Also provided herein are computer-implemented methods for determining plant nitrogen status of a plant, the methods including obtaining, from a chlorophyll sensor, a chlorophyll content of a plant, and obtaining, from one or more ranging sensors, a plurality of data points representing a structure of a plant. The methods also include providing the plurality of data points to a trained machine-learning model, the machine-learning model trained to generate an estimate of one or more parameters related to a biomass of the plant. The methods further include determining, by one or more processing devices based on an output of the trained machine-learning model generated in response to the machine-learning model receiving the plurality of data points, the biomass of the plant, and determining, by the one or more processing devices, plant nitrogen status of the plant based on the chlorophyll content and the biomass of the plant.

Also provided herein are system for determining plant nitrogen status of a plant, the systems including memory and one or more processing devices coupled to the memory. The one or more processing devices are configured to execute machine-readable instruction to perform operations that include obtaining, from a chlorophyll sensor, a chlorophyll content of a plant, and obtaining, from one or more ranging sensors, a plurality of data points representing a structure of a plant. The operations also include providing the plurality of data points to a trained machine-learning model, wherein the machine-learning model is trained to generate an estimate of one or more parameters related to a biomass of the plant. The operations further include determining, by the one or more processing devices based on an output of the trained machine-learning model generated in response to the machine-learning model receiving the plurality of data points, the biomass of the plant, and determining, by the one or more processing devices, plant nitrogen status of the plant based on the chlorophyll content and the biomass of the plant.

Also described herein are one or more non-transitory machine-readable storage devices storing instructions that are executable by one or more processing devices, and upon such execution cause the one or more processing devices to perform various operations. The operations include obtaining, from a chlorophyll sensor, a chlorophyll content of a plant, and obtaining, from one or more ranging sensors, a plurality of data points representing a structure of a plant. The operations also include providing the plurality of data points to a trained machine-learning model, wherein the machine-learning model trained to generate an estimate of one or more parameters related to a biomass of the plant. The operations further include determining, by the one or more processing devices based on an output of the trained machine-learning model generated in response to the machine-learning model receiving the plurality of data points, the biomass of the plant, and determining, by the one or more processing devices, plant nitrogen status of the plant based on the chlorophyll content and the biomass of the plant.

Embodiments of the above aspects can include one or more of the following features. Determining the biomass of the plant based on the output of the trained-machine-learning model can include obtaining, from the trained machine-learning model as the one or more parameters related to the biomass of the plant, a first estimate of volume of the plant and a second estimate of a height of the plant, and determining the biomass of the plant based on the first and second estimates. The one or more ranging sensors can include at least one Light Detection and Ranging (LiDAR) sensor. The one or more ranging sensors can be disposed on a mobile device. The one or more ranging sensors can be disposed on an unmanned aerial vehicle (UAV). The one or more ranging sensors can be disposed on a land vehicle. The one or more parameters related to a biomass of the plant can include one or more of: stem diameter, plant volume, plant height, or leaf area index. In some embodiments, a signal can be generated based on the determined nitrogen status of the plant, and the signal can be used to actuate an agricultural dispensing system to dispense one or more substances that affect the nitrogen status. The one or more substances can include at least one of: a fertilizer, a nitrogen stabilizer, a nitrification inhibitor, a urease inhibitor, or a microbe. The determined nitrogen status of the plant can be stored in a database, wherein the nitrogen status of the plant is linked within the database to field characterization data associated with the plant. The field characterization data can include at least one of: precipitation data, temperature data, field boundaries data, soil type data, or fertility plan data. The plant can be treated with an alternative nitrogen treatment. The alternative nitrogen treatment can include a nitrogen fixing microbe. In some embodiments, the nitrogen fixing microbe can include a microbe selected from Table 1 provided below. In some embodiments, the determined plant nitrogen status can be used to validate the provision of nitrogen to the plant by the alternative nitrogen treatment.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed.

As used herein, the term “about” is used synonymously with the term “approximately.” Illustratively, the use of the term “about” with regard to an amount indicates that values slightly outside the cited values, e.g., plus or minus 0.1% to 10%.

As used herein the term “plant” can include plant parts, tissue, leaves, roots, root hairs, rhizomes, stems, seeds, ovules, pollen, flowers, fruit, etc.

As used herein the terms “microorganism” or “microbe” should be taken broadly. These terms, used interchangeably, include but are not limited to, the two prokaryotic domains, Bacteria and Archaea. The term may also encompass eukaryotic fungi and protists.

The term “determining” encompasses a wide variety of actions and, therefore, “determining” can include calculating, computing, processing, deriving, investigating, looking up (e.g., looking up in a table, a database or another data structure), ascertaining and the like. “Determining” also can include receiving (e.g., receiving information), accessing (e.g., accessing data in a memory) and the like. “Determining” further can include resolving, selecting, choosing, establishing and the like. “Determining” can also include measuring a value.

The phrase “based on” does not mean “based only on,” unless expressly specified otherwise. In other words, the phrase “based on” describes both “based only on” and “based at least on.”

No language in the specification should be construed as indicating any non-claimed element as essential to the practice of the disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWING

FIGS. 1A-1F are graphs of various plant measurements for maize plants treated with a grower standard nitrogen fertilization or treated with nitrogen fixing microbial(s) and a reduction of 35 lbs of synthetic nitrogen (N). FIG. 1A is a graph of plant fresh weight in lbs per plant for treated maize plants. FIG. 1B is a graph of leaf chlorophyll concentration in pmol per m² for treated maize plants. FIG. 1C is a graph of plant nitrogen update in lbs of nitrogen (N) per acre for treated maize plants. FIG. 1D is a graph of plant biomass in lbs per plant for treated maize plants. FIG. 1E is a graph of plant nitrogen concentration as a percentage for treated maize plants. FIG. 1F is a graph of plant nitrogen uptake in lbs nitrogen (N) per acre for treated maize plants.

FIG. 2 is a block diagram of an example system that can be used to implement the technology described herein.

FIG. 3 is a flowchart of an example set of operations performed to determine plant nitrogen status of a plant.

FIG. 4 shows block diagrams of example computing devices that can be used to implement the technology described herein.

FIG. 5 is a graph plotting the relationship (Pearson correlation) between plant nitrogen uptake as determined by laboratory combustion analysis (y-axis) and in-field analysis (x-axis) during the V8-V18 corn growth stages.

FIG. 6 is a graph plotting plant nitrogen uptake (kg nitrogen ha⁻¹) in 2022 (n=575). Treated plants included plants that had a 35 to 40 lb reduction in synthetic nitrogen and were treated with Pivot Bio PROVEN® 40 (PROVEN® 40 replacing 35 lbs N), and plants treated with the grower standard nitrogen practice (Grower Standard Practice). Data are represented as mean±standard error.

### DETAILED DESCRIPTION

This document provides easy and scalable methods for real-time, comparative estimation of plant nitrogen status of one or more plants. The plant nitrogen status can be used, for example, to calculate a relative comparison of plant nitrogen status across, for example, sections of a field or different fields. Plants in these different sections of the field or in different fields may be subjected to different nitrogen management treatments, for example, plants from one field or one section of a field may be subjected to nitrogen fixing microbes whereas plants from another section of the field or from a different field may be subjected to synthetic nitrogen fertilizer.

There are currently limited methods for estimating relative whole plant nitrogen content at the field scale between nutrient management practices. Chlorophyll meters alone have been used to compare relative plant nitrogen status, not plant nitrogen content (see, for example, Penn State Extension. 2008. Agronomy Facts 53: The Early Season Chlorophyll Meter Test for Corn). However, these tests require a high nitrogen reference field plot, which is not practical in commercial settings. Additionally, current techniques for assessing plant nitrogen content requires laboratory analysis of plants for nitrogen concentration (see, for example, Miniat et al. Manual: Procedures for Chemical Analysis. Coweeta Hydrologic Laboratory or Zimmerman et al. 1997. Method Manual: Determination of Carbon and Nitrogen in Sediments and Particulates of Estuarine/Coastal Waters Using Elemental Analysis), but these methods are time consuming and costly. There is need in the art for easy, fast, and scalable methods of assessing plant nitrogen status in the field.

The methods described herein can include determining the chlorophyll content and biomass of a plant or a plurality of plants identified in a field or a first region of a field. The chlorophyll content and biomass can be normalized, and plant nitrogen status can be determined using the normalized content (CC) and the normalized plant biomass (PB).

The methods described herein can be used, for example, to make a relative comparison of plant nitrogen status between different regions or subsections of a field or between different fields with, for example, differing nutrient management practices. For example, the amount of plant nitrogen in a control field or subsection of a field can be compared to that of a different field or subsection of a field where, for example, a synthetic nitrogen fertilizer was applied or, for example, where biological nitrogen fixing microorganisms were applied, optionally where biological nitrogen fixing microorganisms were applied with less synthetic nitrogen (e.g., less fertilizer) than typically used. Such an analysis can be used, for example, to demonstrate the success of such an alternative nutrient management practice.

Comparisons among different fields or among different subsections of a field can use a comparison of plant nitrogen status. Determining plant nitrogen status of a plant can include determining the chlorophyll content of a plant of a plurality of plants identified in a field or a first region of a field; determining the biomass of the plant; normalizing the determined biomass and the determined chlorophyll content for the plant; and determining plant nitrogen status of the plant using the normalized chlorophyll content (CC) and the normalized plant biomass (PB).

### Pluralities of Plants

In some embodiments, a plant of a plurality of plants is used to measure chlorophyll content, plant biomass, or both. In some embodiments, multiple plants (e.g., 2, 3, 4, 5, 6, 7, 8, 9, 10, 11, 12, 13, 14, 15, 16, 17, 18, 19, or 20 plants) of a plurality of plants are used to measure chlorophyll content, plant biomass, or both. In some embodiments, the chlorophyll content of each plant in a plurality of plants is measured.

In some embodiments, a plant of an additional plurality of plants (e.g., in a different field, or a different subsection or location of the same field) is used to measure chlorophyll content, plant biomass, or both. In some embodiments, multiple plants (e.g., 2, 3, 4, 5, 6, 7, 8, 9, 10, 11, 12, 13, 14, 15, 16, 17, 18, 19, or 20 plants) of the additional plurality of plants (e.g., in a different field, or a different subsection or location of the same field) are used to measure chlorophyll content, plant biomass, or both. In some embodiments, the chlorophyll content of each plant in the additional plurality of plants (e.g. in a different field, or a different subsection or location of the same field) is measured. In some embodiments, the additional plurality of plants is from a different field than the plurality of plants. In some embodiments, the plurality of plants is from a first region of a field and the additional plurality of plants is from a second region of the field.

In some embodiments, the multiple plants in a plurality of plants are of the same type of plant (e.g., same species of plant, same crop). In some embodiments, the multiple plants in a plurality of plants are of different types of plants (e.g., different species of plants, different crops). In some embodiments, each plant in a plurality of plants is of the same plant (e.g., same species of plant, same crop). In some embodiments, each plant in a plurality of plants is of different types of plants (e.g., different species of plants, different crops).

In some embodiments, the multiple plants in an additional plurality of plants (e.g. in a different field, or a different subsection or location of the same field) are grown in the same soil type. Non-limiting soil types include sandy soil, clay soil, silt soil, peat soil, chalk soil, and loam soil. Soil types can differ in, for example, the identity and proportion of organic (e.g., decomposed leaf litter) and inorganic matter (e.g., minerals) of the soil and the pH. The additional plurality of plants found in a different subsection or location in the same field or in a different field as the first plurality of plants can be grown in soils that have experienced different nutrient management practices, soil amendments (e.g. synthetic nitrogen fertilizer amendments), or microbial amendments (e.g., addition of nitrogen-fixing microbes).

In some embodiments, the additionally plurality of plants can be in a location (e.g., subsection of the same field or different field) that has the same crop growth history (e.g., the same crop rotation practices were used). In some embodiments, the additional plurality of plants can be in a location (e.g., subsection of the same field or different field) that has different crop growth history (e.g., different crop rotation practices were used).

In some embodiments, the plurality of plants or additional plurality of plants comprises between 2 and 10,000 plants, for example, between 4 and 10,000 plants, between 6 and 10,000 plants, between 8 and 10,000 plants, between 10 and 10,000 plants, between 12 and 10,000 plants, between 2 and 1,000 plants, between 4 and 1,000 plants, between 6 and 1,000 plants, between 8 and 1,000 plants, between 10 and 1,000 plants, between 12 and 1,000 plants, between 2 and 100 plants, between 4 and 100 plants, between 6 and 100 plants, between 8 and 100 plants, between 10 and 100 plants, between 12 and 100 plants, between 2 and 50 plants, between 4 and 50 plants, between 6 and 50 plants, between 8 and 50 plants, between 10 and 50 plants, between 12 and 50 plants, between 2 and 25 plants, between 4 and 25 plants, between 6 and 25 plants, between 8 and 25 plants, between 10 and 25 plants, between 12 and 25 plants, between 2 and 20 plants, between 4 and 20 plants, between 6 and 20 plants, between 8 and 20 plants, between 10 and 20 plants, between 2 and 10 plants, between 4 and 10 plants, between 6 and 10 plants, between 2 and 8 plants, between 4 and 8 plants, between 2 and 6 plants, between 4 and 6 plants, or between 2 and 4 plants. In some embodiments, a plurality of plants and/or an additional plurality of plants includes 2, 3, 4, 5, 6, 7, 8, 9, 10, 11, 12, 13, 14, 15, 16, 17, 18, 19, 20, 21, 22, 23, 24, 25, 26, 27, 28, 29, or 30 plants. In some embodiments, the plurality of plants or additional plurality of plants comprises at least 6 plants. In some embodiments, the plurality of plants or additional plurality of plants comprises at least 12 plants.

### Measuring Chlorophyll

In any of the methods described herein, the chlorophyll content of a plant can be determined, for example, with a chlorophyll meter. Chlorophyll includes several related green pigments found in the choloroplasts of plants and algae. It is an essential component of photosynthesis, allowing plants to make energy from light. Chlorophylls absorb light most strongly in the blue and red portion of the electromagnetic spectrum. Multiple types of chlorophyll exist in plants, including chlorophyll a, b, c1, c2, d, and f.

Without wishing to be bound by theory, the measurement of chlorophyll content in a laboratory without a chlorophyll meter is affected by the solvent used to extract the chlorophyll from the plant tissue or material. For example, in diethyl ether, chlorophyll a has approximate absorbance maxima of 430 nm and 662 nm, while chlorophyll b has approximate maxima of 453 nm and 642 nm. See, for example, Porra et al., Biochim. Biophys. Acta 975 (3): 384-394, 1989.

One way the concentration of chlorophyll within the plant tissue can be estimated is by extrapolating from a measurement of the absorption of light in, for example, the near red, red, and far red regions. This can be completed, for example, with a chlorophyll meter. Ratio fluorescence emission can be used to measure chlorophyll content. By exciting chlorophyll a fluorescence at a lower wavelength, the ratio of chlorophyll fluorescence emission at 705±10 nm and 735±10 nm can provide a linear relationship of chlorophyll content when compared with chemical testing. The ratio F₇₃₅/F₇₀₀ provided a correlation value of r² 0.96 compared with chemical testing in the range from 41 mg m⁻² up to 675 mg m⁻². See, for example, Gitelson et al., Remote Sens. Environ. 69(3):296-302, 1999. Chlorophyll measurements can be in pmol of chlorophyll per m² of plant tissue. Chlorophyll meters, including handheld and portable chlorophyll meters are commercially available, including, for example, from Apogee Instruments, AgTec, and Minolta.

In some embodiments, the chlorophyll content of a plant of a plurality of plants, multiple plants of a plurality of plants, or each plant of a plurality of plants identified in a field or a first region of a field is measured, for example, using a chlorophyll meter. In some embodiments, the chlorophyll content of a plant of an additional plurality of plants, multiple plants of an additional plurality of plants, or each plant of an additional plurality of plants identified in a field or a first region of a field is measured, for example, using a chlorophyll meter.

For example, a chlorophyll meter or chlorophyll sensor can be used to measure the chlorophyll content in a specific leaf (e.g., the uppermost leaf) of a plant of a plurality of plants, of multiple plants of a plurality of plants, or of each plant of a plurality of plants. In some cases, multiple measurements in a single leaf of a single plant are taken, and in some cases averaged together. For example, the chlorophyll content can be measured 1, 2, 3, 4, 5, 6, 6, or 8 times within a single leaf. In some cases, the chlorophyll content can be measured 4 times within a single leaf. In some cases, the 4 chlorophyll content measurements of a single leaf are averaged to determine the chlorophyll content of a plant.

### Measuring Plant Biomass

In some embodiments, the plant biomass, biomass of multiple individual plants of a plurality of plants, or biomass of each plant of a plurality of plants is determined, for example, using a scale (e.g., a digital scale). In some embodiments, a plant of a plurality of plants, multiple plants in a plurality of plants, or biomass of each plant of a plurality of plants is harvested and measured individually, for example, on a digital scale. In some embodiments, the plant biomass, individual biomass of a plurality of plants, or biomass of each plant of a plurality of plants can be estimated such that the plant or multiple plants of plurality of plants are not harvested (i.e., the plant(s) are not destructively sampled).

In some embodiments, a plant of a plurality of plants identified in a field or a first region of a field has its biomass determined. In some embodiments, multiple plants of a plurality of plants identified in a field or a first region of a field has the biomass determined. In some embodiments, each plant of a plurality of plants identified in a field or a first region of a field has the biomass determined.

In some embodiments, the plant biomass, biomass of multiple individual plants of an additional plurality of plants, or biomass of each plant of an additional plurality of plants is determined, for example, using a digital scale. In some embodiments, the plant, multiple plants in an additional plurality of plants, or biomass of each plant of an additional plurality of plants is harvested and measured individually, for example, on a digital scale. In some embodiments, the plant biomass, individual biomass of an additional plurality of plants, or biomass of each plant of an additional plurality of plants can be estimated such that the plant or multiple plants of plurality of plants are not harvested (i.e., the plant(s) are not destructively sampled).

In some embodiments, a plant of an additional plurality of plants identified in a field or a second region of a field has the biomass determined. In some embodiments, multiple plants of an additional plurality of plants identified in a field or a first region of a field has the biomass determined. In some embodiments, each plant of an additional plurality of plants identified in a field or a first region of a field has the biomass determined.

### Examples of Particular Computer-Implemented Systems

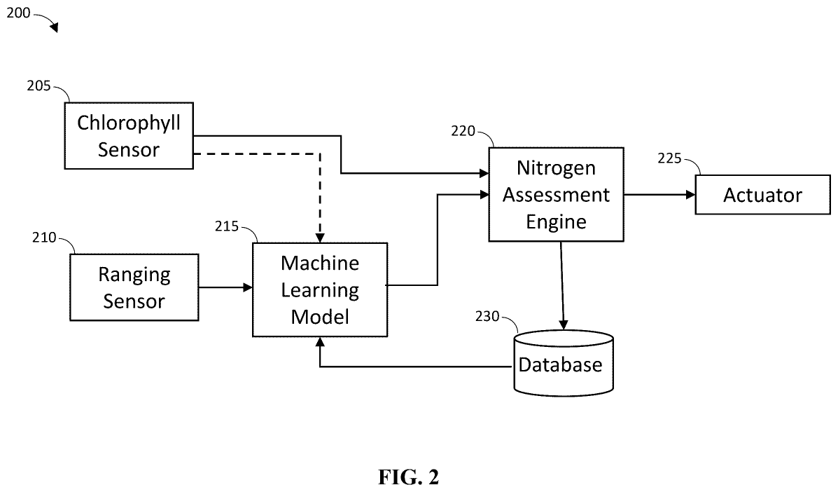

FIG. 2 is a clock diagram of a computer-implemented system 200 that can be used to realize the technology described herein. The system can include one or more chlorophyll sensors 205 (also referred herein as chlorophyll meter) configured to measure the chlorophyll content of a plant or a portion of the plant. In some implementations, the system 200 also includes one or more ranging sensors 205 that can be used to determine the biomass of a plant. Ranging sensors 210 are configured to detect objects without physical contact with the objects. Examples of ranging sensors 210 can include, for example, light detection and ranging (LiDAR) sensors, radio detection and ranging (Radar) sensors, sonic sensors (e.g., Sonar) that use sound waves such as ultrasonic waves for detecting objects, etc. The description below uses the example of LiDAR sensors to illustrate how ranging sensors 210 can be used to detect the biomass of a plant. The concept can be extended to other ranging sensors without deviating from the scope of the technology described herein.

In some implementations, data obtained using LiDAR sensors can be used to form a 3D representation of a structure of a plant using a point cloud of reflected light. The LiDAR device could be a stand-alone detection unit or could be attached to or incorporated in an instrument such as a computer, a cellular device, or a vehicle such as land vehicle or an unmanned aerial vehicle (UAV). The LiDAR measurement can be done on an individual plant, or across a group or field of plants. In some implementations, the point cloud obtained using ranging sensors 205 such as LiDAR can be used in object detection and feature extraction—for example to identify a structure of a plant or a portion thereof. In some implementations, the point cloud can be captured with a stationary detector from a fixed position or by a moving detector such as a LiDAR sensor deployed on a vehicle. Examples of a stationary systems configured to house a LiDAR device can include tripods, mounted poles, or other means to hold the LiDAR device in a fixed position during data capture. Examples of a moving LiDAR device can include a handheld mobile device such as a cellphone or tablet computer, vehicles moving on wheels—potentially on tracks, an UAV such as a fixed wing drone or copter drone, a manned aircraft, or a satellite.

In some implementations, the outputs of the ranging sensors 210 can be used to compute spatial and structural parameters of at least a portion of the captured data, and the spatial and structural parameters (e.g., collection of points representing a structure of a plant) can be provided to a machine learning model to obtain a classification of the portion. For example, spatial and structural parameters extracted from the outputs of the ranging sensors 210 can be preprocessed using one or more processing devices (not shown) and the spatial and structural parameters can be provided to a machine learning model 215 trained to generate an estimate of one or more parameters related to a biomass of a plant. The one or more parameters related to a biomass of the plant can include, for example, stem diameter, plant volume, plant height, or leaf area index—to name a few. In some implementations, the machine-learning model 215 is configured to directly generate an estimate of a nitrogen content of a plant based on, for example, inputs from both the ranging sensors 210 and the chlorophyll sensors 205.

The system 200 can also include a nitrogen assessment engine 220 configured to compute a nitrogen status of a plant based on the outputs of the chlorophyll sensors 205 and the machine-learning model 215. In some implementations, the machine-learning model can be configured to generate an estimate of volume of a plant and an estimate of a height of the plant—from which the biomass of the plant can be computed—and the nitrogen assessment engine 220 can be configured to compute a nitrogen status of the plant based on the chlorophyll content and biomass of the plant, for example, as described elsewhere in this document.

In some implementations, the system 200 includes one or more actuators 225 configured to trigger one or more systems based on the determined nitrogen status of the plant. For example, the nitrogen assessment engine 220 can be configured to generate, based on the determined nitrogen status of the plant, a signal for the one or more actuators 225 to trigger an agricultural dispensing system. In some examples, the agricultural dispensing system can be a fertilizer dispenser that is triggered by the actuator 225—based on the signal received from the nitrogen assessment engine 220—to dispense, increase, or reduce an amount of fertilizer for the plant (or plants) whose nitrogen status has been assessed. In other examples, the agricultural dispensing system can be configured to dispense one or more of a nitrogen stabilizer, a nitrification inhibitor, an urease inhibitor, a microbe (including microbes discussed in this document), or other substances that potentially affect the nitrogen status of plants. In some implementations, the nitrogen assessment engine 220 and the actuator 225 can be located at remote locations with respect to one another, and can be connected, for example over a wired or wireless network such as a LAN, WAN, or the Internet. For example, the actuator 225 can be associated with an Internet-of-Things (IoT) device that the nitrogen assessment engine 220 is configured to trigger based on the determined nitrogen status of plant(s).

In some implementations, the system 200 can include a database 230 that is configured to store the determined nitrogen status of plants. The database can also be configured to link the determined nitrogen status to various field characterization data representing one or more environmental and other conditions associated with the plants. The field characterization data can include, for example, precipitation data, temperature data, field boundaries data, soil type data, or fertility plan data associated with the plants for which the nitrogen status is determined. In some implementations, the data stored in the database 230 can be provided to the machine-learning model 215 as feedback or additional training data, for example, to fine-tune the training of the machine-learning model 215 or even to retrain the machine-learning model.

### Determining Plant Nitrogen Status

In some embodiments, the plant nitrogen status of 1) a plant of a plurality of plants, multiple plants of a plurality of plants, or each plant of a plurality of plants, and/or 2) a plant of an additional plurality of plants, multiple plants of an additional plurality of plants, or each plant of an additional plurality of plants is determined using Formula I. Plant Nitrogen Status (NS)=α(PB)+β(CC), where α and β are scaling factors. Scaling factor α can be a value between 0.50 and 0.90 and scaling factor β can be a value between 0.10 and 0.50. In some embodiments, α is 0.60, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, or 0.90. In some embodiments, α is 0.80. In some embodiments, β is 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, or 0.40. In some embodiments, β is 0.20.

In some embodiments, the method includes normalizing the biomass and the chlorophyll content for a plant of a plurality of plants, multiple plants of a plurality of plants, or each plant of a plurality of plants.

The nitrogen status of a plant of a plurality of plants, multiple plants of a plurality of plants, or each plant of a plurality of plants (NS1) in a field (e.g., a first field) or a subsection of a field (e.g., a first subsection of a field) can be compared to the nitrogen status of a plant of an additional plurality of plants, multiple plants of an additional plurality of plants, or each plant of an additional plurality of plants (NS2) in another field such as a different (e.g., second) field or a different (e.g., second) subsection of the same field as the plurality of plants are grown.

In some embodiments, the method further comprises determining the relative nitrogen status (RNS) of the plurality of plants as compared to the additional plurality of plants. In some embodiments, the RNS can be determined by comparing the NS1 to the average nitrogen status of the plants of the additional plurality of plants (NS2). In some embodiments, the relative nitrogen status is determined using Formula II:

$$Relative Nitrogen Status = ( NS 1 / NS 2 ) - 1$$

In some embodiments, nitrogen performance index (NPI) can be determined. For example, the NPI can be determined using Formula III:

$$NPI = ( Relative Nitrogen Status ) \times 100 \%$$

In some embodiments, plant nitrogen content per acre can be determined. For example, the plant nitrogen content per acre can be determined using the relative nitrogen status and the estimated uptake of nitrogen by growth stage. Non-limiting examples of plant growth stage include seed germination, vegetative growth, reproduction, flowering, and fruit production. For methods of estimating nitrogen uptake by growth stage, for example, in corn, see, for example, Abendroth et al., 2011. Corn growth and development. Iowa State University Extension. PMR 1009.

Successful performance of a different nitrogen management practice may be established depending on the calculated RNS and/or NPI. Non-limiting examples of different nutrient management practices that may be validated based on the calculated RNS and/or NPI include fertilization (e.g., synthetic nitrogen) and/or successful application of nitrogen-fixing microbes to the plant tissues, plant roots, or soil near the plant (e.g., within two meters of the plant). In some embodiments, a plurality of plants determined to have a decreased NS1 as compared to NS2, indicates that the population of plants (or the field or subsection of the field where the population of plants was obtained) were subjected to an unsuccessful or less successful nitrogen management practice. In some embodiments, a plurality of plants determined to have an increased NS1 as compared to NS2, indicates that the additional population of plants (or the field or subsection of the field where the additional population of plants was obtained) received a less successful nitrogen management practice. In such embodiments, the methods described herein can further include discontinuation of the nitrogen management practice with the less successful nitrogen management practice or replacement of the less successful nitrogen management practice with a different nitrogen management practice. In some embodiments, the methods described herein can further include instructing the discontinuation of the nitrogen management practice with the less successful nitrogen management practice or replacement of the less successful nitrogen management practice.

In some embodiments, a plurality of plants determined to have an increased NS1 as compared to NS2, indicates that the population of plants (or the field or subsection of the field where the population of plants was obtained) received a more successful nitrogen management practice. In such embodiments, the methods described herein can further include continuation of the more successful nitrogen management practice or increasing the total number of plants cultivated using the more successful nitrogen management practice. In some embodiments, the methods described herein can further include instructing the continuation of the more successful nitrogen management practice or increasing the total number of plants cultivated using the more successful nitrogen management practice.

In any of the embodiments described herein, the methods can be used for validating the performance of alternative plant nitrogen treatments such as validating the performance of nitrogen fixing microbes in replacing a defined amount of nitrogen from synthetic nitrogen treatment.

In any of the embodiments described herein, the method further can include using the results of the comparison to validate or deny a claim for compensation under a performance guarantee program. For example, if plants receiving a nitrogen fixing microbe treatment perform similar to or better than plants receiving a synthetic nitrogen treatment, compensation may be denied. Alternatively, if plants receiving a nitrogen fixing microbe treatment perform worse than the plants receiving a synthetic nitrogen treatment, compensation may be granted. Alternatively, compensation may be determined at various thresholds of performance, e.g., where the results of the comparison evidence successful replacement of defined amounts (e.g., pounds/acre) of synthetic nitrogen by the nitrogen fixing microbes.

In some embodiments, the methods described herein can include determining the nitrogen fixing capacity of applied or supplemented nitrogen-fixing microbes. The methods described herein can be used to determine the nitrogen status of plants that have been supplemented, for example, with nitrogen fixing dormant microbes (e.g., dormant microbes found in packaged microbial products) that were applied to a plant or soil or used to supplement a synthetic fertilizer.

In some cases, the dormant microbes can become viable when applied to the plants or soil. The microbial products can include any type of microbe, including bacteria and yeast, that is naturally occurring or genetically engineered. In some embodiments, the nitrogen fixing capacity of a microbial product containing Agrobacterium radiobacter, Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus) Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus licheniformis, Bacillus maroccanus, Bacillus megaterium, Bacillus metiens, Bacillus mycoides, Bacillus natto, Bacillus nematocida, Bacillus nigrificans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus unflagellatus, Bradyrhizobium japonicum, Brevibacillus brevis Brevibacillus laterosporus (formerly Bacillus laterosporus), Chromobacterium subtsugae, Delftia acidovorans, Klebsiella variicola, Kosokonia sacchari, Lactobacillus acidophilus, Lysobacter antibioticus, Lysobacter enzymogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorum (formerly Erwinia carotovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luminescens, Xenorhabdus nematophila, Rhodococcus globerulus AQ719 (NRRL Accession No. B-21663), Bacillus sp. AQ175 (ATCC Accession No. 55608), Bacillus sp. AQ 177 (ATCC Accession No. 55609), Bacillus sp. AQ178 (ATCC Accession No. 53522), or Streptomyces sp. strain NRRL Accession No. B-30145, or any combination thereof, can be determined. In some embodiments, the nitrogen fixing capacity can be determined of a microbial product containing Azotobacter chroococcum, Methanosarcina barkeri, Klesiella pneumoniae, Azotobacter vinelandii, Rhodobacter spharoides, Rhodobacter capsulatus, Rhodobcter palustris, Rhodosporillum rubrum, Rhizobium leguminosarum, or Rhizobium etli, or any combination thereof.

In some embodiments, the nitrogen fixing capacity can be determined of a microbial product containing cyanobacteria such as a species from Anabaena (for example Anagaena sp. PCC7120), Nostoc (for example Nostoc punctiforme), or Synechocystis (for example Synechocystis sp. PCC6803), or any combination thereof.

In some embodiments, the methods provided herein can be used to determine the nitrogen fixing capacity of applied or supplemented genetically engineered bacteria that comprise at least one modification in a gene regulating nitrogen fixation or assimilation. For example, the methods provided herein can be used determine the nitrogen fixing capacity of one or more applied or supplemented genetically engineered strains of Rahnella aquatilis, Kosakonia sacchari, Kosakonia arachidis, Klebsiella variicola, Paraburkholderia tropica, Herbaspirillum seropedicae, Herbaspirillum aquaticum, and Paenibacillus polymyxa, wherein each comprise at least one modification in a gene regulating nitrogen fixation or assimilation. See, e.g., WO2021221690A1, filed May 1, 2020, WO2021222567A2, filed Apr. 29, 2021, and U.S. Provisional Application No. 63/220,313, filed Jul. 9, 2021. In some embodiments, the methods described herein can be used to determine the nitrogen fixing capacity of an applied or supplemented Kosakonia sacchari strain identified by American Type Culture Collection (ATCC) Accession number PTA-126743 and the nitrogen fixing capacity of an applied or supplemented Klebsiella variicola strain identified by ATCC Accession No. PTA-126740 or the PROVEN40 product containing a combination of the microbes. In some embodiments, the methods described herein can be used to determine the nitrogen fixing capacity of an applied or supplemented Kosakonia sacchari strain identified by ATCC deposit number PTA-126743, a Kosakonia arachidis strain that is a genetically engineered form of a bacterium deposited as LMG 26131 (e.g., Kosakonia arachidis strain 1661-5402 and having the genotype ΔnifL_PompX_v2-nifA ΔglnD glnE_ΔAR), or a Paraburkholderia tropica strain that is a genetically engineered form of a bacterium deposited as PTA-126582 (e.g., strain 8-5063 and having the genotype P(rpsL)-nifA_Δgaf_v3, glnD_ΔUTase). Table 1 lists the deposit information for exemplary strains deposited with National Center for Marine Algae and Microbiota (NCMA) or ATCC. Each of the deposits was made under the provisions of the Budapest Treaty on the International Recognition of the Deposit of Microorganisms for the Purpose of Patent Procedure and the Regulations (Budapest Treaty).

TABLE 1
Microorganisms Deposited under the Budapest Treaty

| | Pivot Strain | | | |
|---|---|---|---|---|
| | Designation | | | |
| | (some strains | | | |
| | have multiple | | Accession | |
| Depository | designations) | Taxonomy | Number | Date of Deposit |
| NCMA | CI006, | Kosakonia sacchari (WT) | 201701001 | Jan. 6, 2017 |
| | PBC6.1, 6 | | | |
| NCMA | CI019, 19 | Rahnella aquatilis (WT) | 201701003 | Jan. 6, 2017 |
| NCMA | CM029, 6-412 | Kosakonia sacchari | 201701002 | Jan. 6, 2017 |
| NCMA | 6-403 | Kosakonia sacchari | 201708004 | Aug. 11, 2017 |
| | CM037 | | | |
| NCMA | 6-404, | Kosakonia sacchari | 201708003 | Aug. 11, 2017 |
| | CM38, | | | |
| | PBC6.38 | | | |
| NCMA | CM094, | Kosakonia sacchari | 201708002 | Aug. 11, 2017 |
| | 6-881, | | | |
| | PBC6.94 | | | |
| NCMA | CI137, 137, | Klebsiella variicola (WT) | 201708001 | Aug. 11, 2017 |
| | PB137 | | | |
| NCMA | 137-1034 | Klebsiella variicola | 201712001 | Dec. 20, 2017 |
| NCMA | 137-1036 | Klebsiella variicola | 201712002 | Dec. 20, 2017 |
| ATCC | 6-2425 | Kosakonia sacchari | PTA-126575 | Dec. 23, 2019 |
| ATCC | 6-2634 | Kosakonia sacchari | PTA-126576 | Dec. 23, 2019 |
| ATCC | 137-1968 | Klebsiella variicola | PTA-126577 | Dec. 23, 2019 |
| ATCC | 137-2219 | Klebsiella variicola | PTA-126578 | Dec. 23, 2019 |
| ATCC | 137-2237 | Klebsiella variicola | PTA-126579 | Dec. 23, 2019 |
| ATCC | 137-2285 | Klebsiella variicola | PTA-126580 | Dec. 23, 2019 |
| ATCC | 41 | Paenibacillus polymyxa | PTA-126581 | Dec. 23, 2019 |
| | | (WT) | | |
| ATCC | 8 | Paraburkholderia tropica | PTA-126582 | Dec. 23, 2019 |
| | | (WT) | | |
| ATCC | 3069 | Herbaspirillum aquaticum | PTA-126583 | Dec. 23, 2019 |
| | | (WT) | | |
| ATCC | 137-2253 | Klebsiella variicola | PTA-126740 | Mar. 25, 2020 |
| ATCC | 137-3896 | Klebsiella variicola | PTA-126741 | Mar. 25, 2020 |
| ATCC | 137-3890 | Klebsiella variicola | PTA-126749 | Mar. 25, 2020 |
| ATCC | 6-5687 | Kosokonia sacchari | PTA-126743 | Mar. 25, 2020 |

### Plant Species

Any of the methods described herein can be used to determine the nitrogen status of any plants that have economic, social and/or environmental value, such as food crops, fiber crops, oil crops, plants in the forestry or pulp and paper industries, feedstock for biofuel production and/or ornamental plants. Non-limiting examples of crop plants include maize, rice, wheat, barley, sorghum, millet, oats, rye triticale, buckwheat, sweet corn, sugar cane, onions, tomatoes, strawberries, and asparagus. For example, plants can be in the genera Hordeum, Oryza, Zea, and Triticeae.

In some examples, any of the methods described herein can be used to determine the nitrogen status of any plants that may be used to produce economically valuable products such as a grain, a flour, a starch, a syrup, a meal, an oil, a film, a packaging, a nutraceutical product, a pulp, an animal feed, a fish fodder, a bulk material for industrial chemicals, a cereal product, a processed human food product, a sugar, an alcohol, and/or a protein.

In some cases, any of the methods described herein can be used to determine the nitrogen status are cereal plants. Non-limiting examples of cereal plants include corn plants, canola plants, sorghum plants, wheat plants, and sunflower plants,

In some cases, any of the methods described herein can be used to determine the nitrogen status of any plants that are important or interesting for agriculture, horticulture, biomass for the production of biofuel molecules and other chemicals, and/or forestry. Some examples of these plants include pineapple, bamboo, banana, coconut, lily, grass peas and grass; and dicotyledonous plants, such as, for example, peas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum bicolor (sorghum, sudan), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp. Sorghum spp., Miscanthus spp., Saccharum spp., Erianthus spp., Populus spp., Secale cereale (rye), Salix spp. (willow), Eucalyptus spp. (eucalyptus), Triticosecale spp. (triticum-25 wheat×rye), Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassava), Lycopersicon esculentum (tomato), Lactuca saliva (lettuce), Musa paradisiaca (banana), Solanum tuberosum (potato), Brassica oleracea (broccoli, cauliflower, brussel sprouts), Camellia sinensis (tea), Fragaria ananassa (strawberry), Theobroma cacao (cocoa), Coffea arabica (coffee), Vitis vinfera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Hordeum vulgare (barley), and Lolium spp. (rye).

In some examples, a monocotyledonous plant may be used. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. For example, the methods described herein can be performed on plant(s) belonging to the class of Gymnospermae are Cycadales, Ginkgoales, Gnetales, and Pinales. In some examples, the monocotyledonous plant can be selected from the group consisting of a maize, rice, wheat, barley, and sugarcane.

In some examples, a dicotyledonous plant may be used, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates.

Other non-limiting examples of suitable plants include mosses, lichens, and algae.

In some embodiments, the methods described herein are suitable for any of a variety of transgenic plants, non-transgenic plants, and hybrid plants thereof.

### Non-Genetically Modified Maize

The methods described herein are suitable for determining the nitrogen status of any of a variety of non-genetically modified maize plants or part thereof. Furthermore, the methods and bacteria described herein are suitable for any of the following non-genetically modified hybrids, varieties, lineages, etc. In some embodiments, corn varieties generally fall under six categories: sweet corn, flint corn, popcorn, dent corn, pod corn, and flour corn.

### Sweet Corn

Yellow su varieties include Earlivee, Early Sunglow, Sundance, Early Golden Bantam, Iochief, Merit, Jubilee, and Golden Cross Bantam. White su varieties include True Platinum, Country Gentleman, Silver Queen, and Stowell's Evergreen. Bicolor su varieties include Sugar & Gold, Quickie, Double Standard, Butter & Sugar, Sugar Dots, Honey & Cream. Multicolor su varieties include Hookers, Triple Play, Painted Hill, Black Mexican/Aztec.

Yellow se varieties include Buttergold, Precocious, Spring Treat, Sugar Buns, Colorow, Kandy King, Bodacious R/M, Tuxedo, Incredible, Merlin, Miracle, and Kandy Korn EH. White se varieties include Spring Snow, Sugar Pearl, Whiteout, Cloud Nine, Alpine, Silver King, and Argent. Bicolor se varieties include Sugar Baby, Fleet, Bon Jour, Trinity, Bi-Licious, Temptation, Luscious, Ambrosia, Accord, Brocade, Lancelot, Precious Gem, Peaches and Cream Mid EH, and Delectable R/M. Multicolor se varieties include Ruby Queen.

Yellow sh2 varieties include Extra Early Super Sweet, Takeoff, Early Xtra Sweet, Raveline, Summer Sweet Yellow, Krispy King, Garrison, Illini Gold, Challenger, Passion, Excel, Jubilee SuperSweet, Illini Xtra Sweet, and Crisp 'N Sweet. White sh2 varieties include Summer Sweet White, Tahoe, Aspen, Treasure, How Sweet It Is, and Camelot. Bicolor sh2 varieties include Summer Sweet Bicolor, Radiance, Honey 'N Pearl, Aloha, Dazzle, Hudson, and Phenomenal. Yellow sy varieties include Applause, Inferno, Honeytreat, and Honey Select. White sy varieties include Silver Duchess, Cinderella, Mattapoisett, Avalon, and Captivate. Bicolor sy varieties include Pay Dirt, Revelation, Renaissance, Charisma, Synergy, Montauk, Kristine, Serendipity/Providence, and Cameo.

Yellow augmented supersweet varieties include Xtra-Tender 1ddA, Xtra-Tender 11dd, Mirai 131Y, Mirai 130Y, Vision, and Mirai 002. White augmented supersweet varieties include Xtra-Tender 3dda, Xtra-Tender 31dd, Mirai 421 W, XTH 3673, and Devotion. Bicolor augmented supersweet varieties include Xtra-Tender 2dda, Xtra-Tender 21dd, Kickoff XR, Mirai 308BC, Anthem XR, Mirai 336BC, Fantastic XR, Triumph, Mirai 301BC, Stellar, American Dream, Mirai 350BC, and Obsession.

### Flint Corn

Flint corn varieties include Bronze-Orange, Candy Red Flint, Floriani Red Flint, Glass Gem, Indian Ornamental (Rainbow), Mandan Red Flour, Painted Mountain, Petmecky, and Cherokee White Flour.

### Pop Corn

Pop corn varieties include Monarch Butterfly, Yellow Butterfly, Midnight Blue, Ruby Red, Mixed Baby Rice, Queen Mauve, Mushroom Flake, Japanese Hull-less, Strawberry, Blue Shaman, Miniature Colored, Miniature Pink, Pennsylvania Dutch Butter Flavor, and Red Strawberry.

### Dent Corn

Dent corn varieties include Bloody Butcher, Blue Clarage, Ohio Blue Clarage, Cherokee White Eagle, Hickory Cane, Hickory King, Jellicorse Twin, Kentucky Rainbow, Daymon Morgan's Knt. Butcher, Learning, Learning's Yellow, McCormack's Blue Giant, Neal Paymaster, Pungo Creek Butcher, Reid's Yellow Dent, Rotten Clarage, and Tennessee Red Cob.

In some embodiments, corn varieties include P1618 W, P1306 W, P1345, P1151, P1197, P0574, P0589, and P0157. (W=white corn.) In some embodiments, the methods described herein are suitable for any hybrid of the maize varieties set forth herein.

### Genetically Modified Maize

The methods and bacteria described herein are suitable for any of a hybrid, variety, lineage, etc. of genetically modified maize plants or part thereof.

### Non-Genetically Modified Sorghum

The methods described herein are suitable for determining the nitrogen status of any of a variety of non-genetically modified sorghum plants or part thereof. Furthermore, the methods and bacteria described herein are suitable for any of the following non-genetically modified hybrids, varieties, lineages, etc.

Sorghum is a genus of plant that includes multiple species of flowering plants in the Poaceae family and is also known as durra, jowari, and milo. In some embodiments, sorghum species include, Sorghum amplum, Sorghum angustum, Sorghum arundinaceum, Sorghum bicolor, Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum nitidum, Sorghum plumosum, Sorghum propinquum, Sorghum purpureosericeum, Sorghum stipoideum, Sorghum timorense, Sorghum trichocladum, Sorghum versicolor, and Sorghum virgatum.

### Genetically Modified Sorghum

The methods and bacteria described herein are suitable for any of a hybrid variety, lineage, etc, of genetically modified sorghum plants or a part thereof, including varieties with genetic modifications to increase grain production, increase plant growth rate, or increase plant yield.

### Example Processes

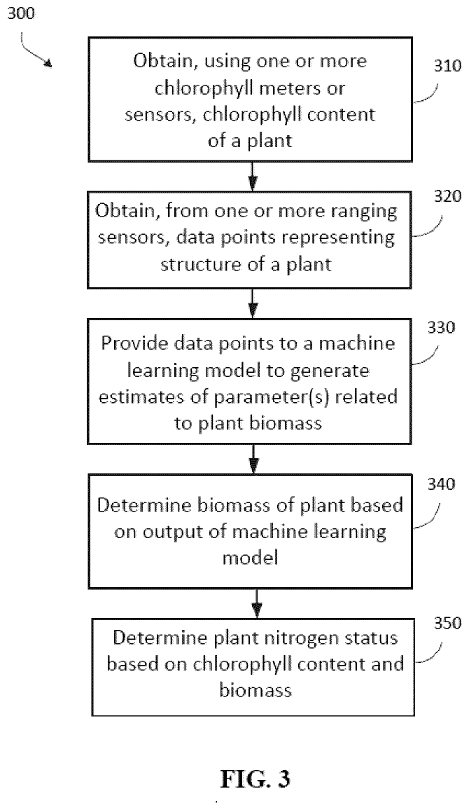

FIG. 3 is a flowchart 300 of an example set of operations performed to determine plant nitrogen status of a plant. In some implementations, at least a portion of the operations depicted in the flowchart 300 can be performed by the various modules described above with references to FIG. 2. For example, the biomass determination and/or the nitrogen status determination can be performed in a module substantially similar to the nitrogen assessment engine 220 described with reference to FIG. 2.

The process represented in the flowchart 300 can include obtaining, from a chlorophyll sensor, a chlorophyll content of a plant (310). In some implementations, the chlorophyll sensor can be substantially similar to the chlorophyll sensor 205 referenced in FIG. 2. The process can also include obtaining, from one or more ranging sensors, a plurality of data points representing a structure of a plant (320). In some implementations, the ranging sensors can be substantially similar to the ranging sensors 210 referenced in FIG. 2. For example, the one or more ranging sensors can include at least one of: a LiDAR sensor, a radar sensor, or a sonar sensor. In some implementations, the one or more ranging sensors can be disposed on a mobile device, or a vehicle such as a land vehicle or a UAV.

The process represented by the flowchart 300 can also include providing the data points to a trained machine-learning model to generate an estimate of one or more parameters related to a biomass of the plant (330). In some implementations, the machine-learning model can be substantially similar to the machine-learning model 215 described with reference to FIG. 2. In some implementations, the machine-learning model can be trained via supervised learning, using a pre-labeled training data set. In some implementations, a convolutional neural network (CNN) or a deep neural network (DNN) can be used as the machine-learning model. In some implementations, the one or more parameters related to a biomass of the plant can include one or more of: stem diameter, plant volume, plant height, or leaf area index.

In some implementations, the process represented in FIG. 3 can include determining, based on an output of the trained machine-learning model, the biomass of the plant (340). The output of the machine-learning model can be generated in response to the machine-learning model receiving the plurality of data points. In some implementations, determining the biomass of the plant based on the output of the trained-machine-learning model can include obtaining, from the trained machine-learning model as the one or more parameters related to the biomass of the plant, an estimate of volume of the plant and an estimate of a height of the plant. The biomass of the plant can then be determined based on such estimates as described in this document.

In some implementations, the process represented by the flowchart 300 can include determining, a plant nitrogen status of the plant based on the chlorophyll content and the biomass of the plant (350). This can be done, for example, by normalizing the biomass and the chlorophyll content, and determining the nitrogen status as a function of the normalized biomass and chlorophyll content, as described herein.

The determined plant nitrogen status can be used for various purposes. In some implementations, a signal may be generated based on the determined plant nitrogen status, the signal being configured to actuate an agricultural dispensing system to dispense one or more substances that affect the nitrogen status. For example, the actuating signal can trigger an agricultural dispensing system to adjust, start or stop dispensing one or more of: a fertilizer, a nitrogen stabilizer, a nitrification inhibitor, an urease inhibitor, or a microbe. In some implementations, the determined nitrogen status of the plant can be stored in a database, wherein the nitrogen status of the plant is linked within the database to field characterization data associated with the plant. The field characterization data can include at least one of: precipitation data, temperature data, field boundaries data, soil type data, or fertility plan data. In some implementations, the determined nitrogen status can be made available to a user (e.g., a farmer) over a user-interface displayed on a computing device or a mobile device. The determined nitrogen status may also be made available as feedback to other associated personnel, e.g., an agronomist, to determine whether a follow-up field visit needs to be scheduled. In some cases, the determined nitrogen status can be used to determine whether any changes in seeding rate or crop selection need to be implemented in subsequent years to improve yield.

The invention will be further described in the following examples, which do not limit the scope of the invention described in the claims.

### EXAMPLES

### Measuring Nitrogen Status and Uptake in Corn Plants in Fields.

Twelve corn plants were harvested from two areas of a field. One treatment area (Treated) in the field received biological nitrogen fixing microorganisms, including strains of Klebsiella variicola and Kosakonia sacchari, and had a 35 lbs reduction of synthetic nitrogen fertilizer compared to the grower's standard practice. The second area of the field (Grower Standard Practice) used grower standard practice of synthetic nitrogen without the addition of biological nitrogen fixing microorganisms. Here, grower standard practices refers to the historical nutrient management plan used by the grower. Grower standard practice can differ between growers, fields, soil types, states, etc. depending on the nutrient needs of the soil and crop. This term is used to represent what the typical, historical nutrient management practice that a grower uses on their field. The biomass of each plant was measured using a digital scale then, the uppermost fully collared leaf from each of the twelve plants was removed. Chlorophyll content (CC) (mol/m²) was measured in the middle of each leaf equal distance between the leaf edge and midrib using a chlorophyll meter (MC-100 Chlorophyll Concentration Meter, Apogee Instruments). This was repeated four times per leaf. The four replicate values per plant were averaged together, resulting in a mean CC for each plant.

Whole plant nitrogen content is a function of total plant biomass and plant organ nitrogen concentrations. Leaf chlorophyll concentration is proxy for leaf nitrogen concentration. In corn, leaves represent the major sink of nitrogen during vegetative growth leading into reproductive growth, when ears become the nitrogen sink. Therefore, plant biomass (PB) and CC were used to estimate relative plant nitrogen status: Nitrogen status=(PB)*(0.80)+β(CC)*(0.20). The scaling factors for corn were determining using an iterative approach testing number scaling factors with a focus on accurate prediction compared to laboratory results.

Estimated nitrogen uptake was subsequently calculated:

(Nitrogen status)*(average nitrogen uptake by corn growth stage).

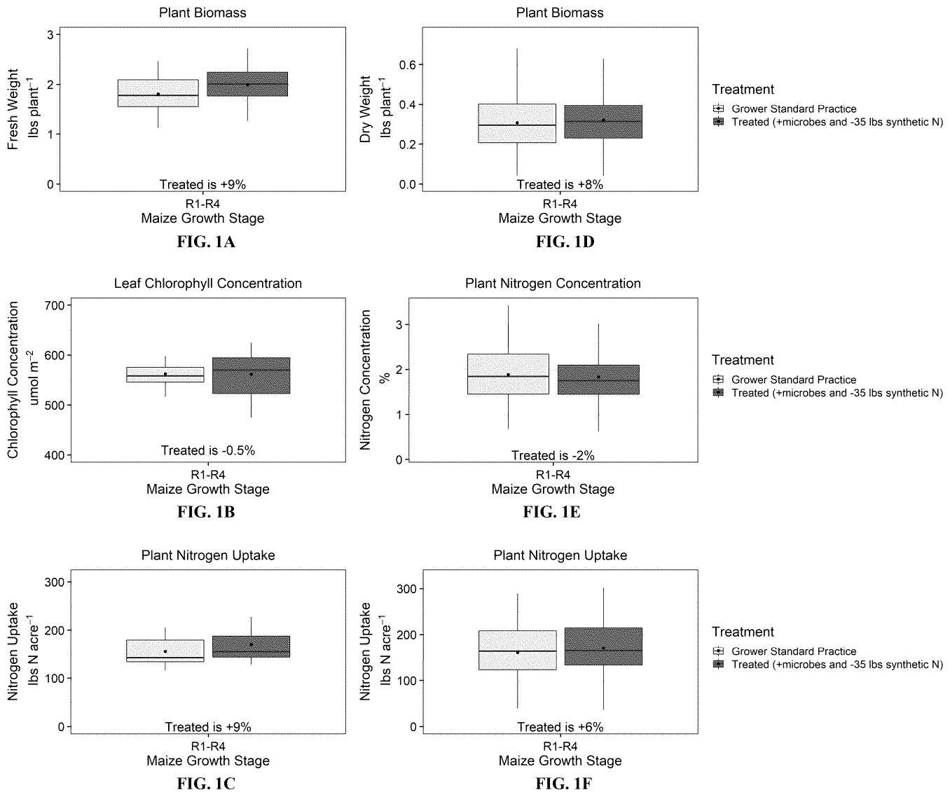

To confirm repeatability, this test was performed at approximately 80 field sites in 13 states in the United States. Plant biomass (FIG. 1A), CC (FIG. 1B) and estimated plant nitrogen uptake (FIG. 1C), as determined with this new test, were similar to results from laboratory analysis using standard nitrogen measurement assay (FIGS. 1D-1F). There was more nitrogen in plants from treated areas relative to Grower Standard Practice areas in 71% (win/loss ratio) of the fields according to the laboratory nitrogen measurement assay. This new relative plant nitrogen assessment test accurately predicted 82% of these win/losses.

### LiDAR-Based Computations

Plant fresh weights and LiDAR data were collected from plants at the V8 stage of growth from seven research trials across the US (63 total replications). In these trials the recommended applied nitrogen was determined using previous crop history and a soil nitrogen analysis, and this full grower recommended nitrogen rate (100% NTC) was applied as a positive control (100% NTC). A reduction of forty pounds per acre of nitrogen from the grower recommended rate (−401b) was used as the negative control. The PROVEN® 40 product was applied as a seed treatment to the same hybrid seeds used across an entire site, and the product was tested at the −401b nitrogen rate (−401b PROVEN® 40).

For plant fresh weights, three plants per plot were collected according to the previously described method. This consisted of clipping the entire plant just above the soil surface and measuring total plant biomass in the field using a handheld scale.

For LiDAR measurements, each plot was covered at approximately the same developmental as the V8 fresh weight measurements using a LiDAR device on a UAV. Point cloud data from the LiDAR device was used to create 3D models of each plot, and various traits known to correlate with plant biomass were generated using a variety of custom analysis scripts run in Python and R. Plant height—as computed by LiDAR—was able to discriminate the full nitrogen rate from the −40 lbs N rate with a p value of 0.0002 using a linear mixed-effects model analysis with either nitrogen rate or microbial application (treatment) as a fixed effect and location and replication as random effects.

A Pearson correlation was also performed using average LiDAR height values for each plot compared to the average plant fresh weight from each plot. The outcome of this analysis showed that fresh weight and height by LiDAR are positively correlated with an R² of 0.25.

### Example Computing Systems

A number of implementations have been described. Nevertheless, it will be understood that various modifications may be made without departing from the spirit and scope of the disclosure. For example, various forms of the flows shown above may be used, with steps re-ordered, added, or removed.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” refers to data processing hardware and encompasses all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program include, by way of example, general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a smart phone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., LCD (liquid crystal display), OLED (organic light emitting diode) or other monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other.

In some embodiments, a server transmits data, e.g., an Hypertext Markup Language (HTML) page, to a user device, e.g., for purposes of displaying data to and receiving user input from a user interacting with the user device, which acts as a client. Data generated at the user device, e.g., a result of the user interaction, can be received from the user device at the server.

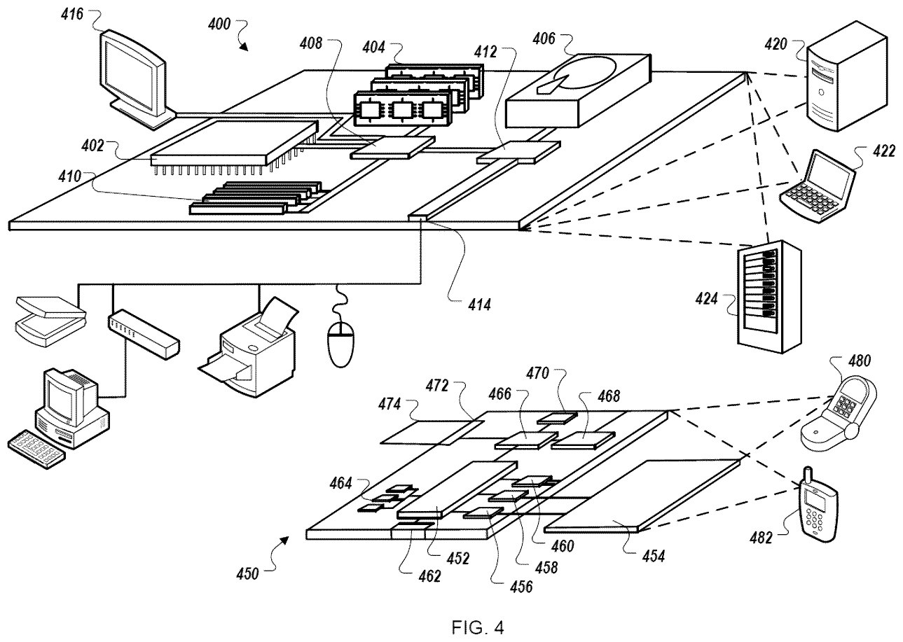

FIG. 4 shows an example of a computing device 400 and a mobile computing device 450 (also referred to herein as a wireless device) that are employed to execute implementations of the present disclosure. For example, the machine learning model 215 and/or the nitrogen assessment engine 220 described above may be implemented, at least in part, on a computing device 400, a mobile computing device 450, or a combination thereof. A computing device 400 or a mobile computing device 450 can include, or can be configured to communicate with, one or more chlorophyll sensors 205 and/or one or more ranging sensors 210 The computing device 400 is intended to represent various forms of digital computers, such as laptops, desktops, workstations, personal digital assistants, servers, blade servers, mainframes, and other appropriate computers. The mobile computing device 450 is intended to represent various forms of mobile devices, such as personal digital assistants, cellular telephones, smart-phones, AR devices, and other similar computing devices. The components shown here, their connections and relationships, and their functions, are meant to be examples only, and are not meant to be limiting.

The computing device 400 includes a processor 402, a memory 404, a storage device 406, a high-speed interface 408, and a low-speed interface 412. In some implementations, the high-speed interface 408 connects to the memory 404 and multiple high-speed expansion ports 410. In some implementations, the low-speed interface 412 connects to a low-speed expansion port 414 and the storage device 404. Each of the processor 402, the memory 404, the storage device 406, the high-speed interface 408, the high-speed expansion ports 410, and the low-speed interface 412, are interconnected using various buses, and may be mounted on a common motherboard or in other manners as appropriate. The processor 402 can process instructions for execution within the computing device 400, including instructions stored in the memory 404 and/or on the storage device 406 to display graphical information for a graphical user interface (GUI) on an external input/output device, such as a display 416 coupled to the high-speed interface 408. In other implementations, multiple processors and/or multiple buses may be used, as appropriate, along with multiple memories and types of memory. In addition, multiple computing devices may be connected, with each device providing portions of the necessary operations (e.g., as a server bank, a group of blade servers, or a multi-processor system).

The memory 404 stores information within the computing device 400. In some implementations, the memory 404 is a volatile memory unit or units. In some implementations, the memory 404 is a non-volatile memory unit or units. The memory 404 may also be another form of a computer-readable medium, such as a magnetic or optical disk.

The storage device 406 is capable of providing mass storage for the computing device 400. In some implementations, the storage device 406 may be or include a computer-readable medium, such as a floppy disk device, a hard disk device, an optical disk device, a tape device, a flash memory, or other similar solid-state memory device, or an array of devices, including devices in a storage area network or other configurations. Instructions can be stored in an information carrier. The instructions, when executed by one or more processing devices, such as processor 402, perform one or more methods, such as those described above. The instructions can also be stored by one or more storage devices, such as computer-readable or machine-readable mediums, such as the memory 404, the storage device 406, or memory on the processor 402.

The high-speed interface 408 manages bandwidth-intensive operations for the computing device 400, while the low-speed interface 412 manages lower bandwidth-intensive operations. Such allocation of functions is an example only. In some implementations, the high-speed interface 408 is coupled to the memory 404, the display 416 (e.g., through a graphics processor or accelerator), and to the high-speed expansion ports 410, which may accept various expansion cards. In the implementation, the low-speed interface 412 is coupled to the storage device 406 and the low-speed expansion port 414. The low-speed expansion port 414, which may include various communication ports (e.g., Universal Serial Bus (USB), Bluetooth, Ethernet, wireless Ethernet) may be coupled to one or more input/output devices. Such input/output devices may include a scanner, a printing device, or a keyboard or mouse. The input/output devices may also be coupled to the low-speed expansion port 414 through a network adapter. Such network input/output devices may include, for example, a switch or router.

The computing device 400 may be implemented in a number of different forms, as shown in the FIG. 4. For example, it may be implemented as a standard server 420, or multiple times in a group of such servers. In addition, it may be implemented in a personal computer such as a laptop computer 422. It may also be implemented as part of a rack server system 424. Alternatively, components from the computing device 400 may be combined with other components in a mobile device, such as a mobile computing device 450. Each of such devices may contain one or more of the computing device 400 and the mobile computing device 450, and an entire system may be made up of multiple computing devices communicating with each other.

The mobile computing device 450 includes a processor 452; a memory 464; an input/output device, such as a display 454; a communication interface 466; and a transceiver 468; among other components. In some implementations, the mobile computing device 450 can include one or more ranging sensors such as LiDAR. The mobile computing device 450 may also be provided with a storage device, such as a micro-drive or other device, to provide additional storage. Each of the processor 452, the memory 464, the display 454, the communication interface 466, and the transceiver 468, are interconnected using various buses, and several of the components may be mounted on a common motherboard or in other manners as appropriate. In some implementations, the mobile computing device 450 may include a camera device(s) (not shown).

The processor 452 can execute instructions within the mobile computing device 450, including instructions stored in the memory 464. The processor 452 may be implemented as a chipset of chips that include separate and multiple analog and digital processors. For example, the processor 452 may be a Complex Instruction Set Computers (CISC) processor, a Reduced Instruction Set Computer (RISC) processor, or a Minimal Instruction Set Computer (MISC) processor. The processor 452 may provide, for example, for coordination of the other components of the mobile computing device 450, such as control of user interfaces (UIs), applications run by the mobile computing device 450, and/or wireless communication by the mobile computing device 450.

The processor 452 may communicate with a user through a control interface 458 and a display interface 456 coupled to the display 454. The display 454 may be, for example, a Thin-Film-Transistor Liquid Crystal Display (TFT) display, an Organic Light Emitting Diode (OLED) display, or other appropriate display technology. The display interface 456 may include appropriate circuitry for driving the display 454 to present graphical and other information to a user. The control interface 458 may receive commands from a user and convert them for submission to the processor 452. In addition, an external interface 462 may provide communication with the processor 452, so as to enable near area communication of the mobile computing device 450 with other devices. The external interface 462 may provide, for example, for wired communication in some implementations, or for wireless communication in other implementations, and multiple interfaces may also be used.

The memory 464 stores information within the mobile computing device 450. The memory 464 can be implemented as one or more of a computer-readable medium or media, a volatile memory unit or units, or a non-volatile memory unit or units. An expansion memory 474 may also be provided and connected to the mobile computing device 450 through an expansion interface 472, which may include, for example, a Single in Line Memory Module (SIMM) card interface. The expansion memory 474 may provide extra storage space for the mobile computing device 450, or may also store applications or other information for the mobile computing device 450. Specifically, the expansion memory 474 may include instructions to carry out or supplement the processes described above, and may include secure information also. Thus, for example, the expansion memory 474 may be provided as a security module for the mobile computing device 450, and may be programmed with instructions that permit secure use of the mobile computing device 450. In addition, secure applications may be provided via the SIMM cards, along with additional information, such as placing identifying information on the SIMM card in a non-hackable manner.

The memory may include, for example, flash memory and/or non-volatile random access memory (NVRAM), as discussed below. In some implementations, instructions are stored in an information carrier. The instructions, when executed by one or more processing devices, such as processor 452, perform one or more methods, such as those described above. The instructions can also be stored by one or more storage devices, such as one or more computer-readable or machine-readable mediums, such as the memory 464, the expansion memory 474, or memory on the processor 452. In some implementations, the instructions can be received in a propagated signal, such as, over the transceiver 468 or the external interface 462.

The mobile computing device 450 may communicate wirelessly through the communication interface 466, which may include digital signal processing circuitry where necessary. The communication interface 466 may provide for communications under various modes or protocols, such as Global System for Mobile communications (GSM) voice calls, Short Message Service (SMS), Enhanced Messaging Service (EMS), Multimedia Messaging Service (MMS) messaging, code division multiple access (CDMA), time division multiple access (TDMA), Personal Digital Cellular (PDC), Wideband Code Division Multiple Access (WCDMA), CDMA2000, General Packet Radio Service (GPRS). Such communication may occur, for example, through the transceiver 468 using a radio frequency. In addition, short-range communication, such as using a Bluetooth or Wi-Fi, may occur. In addition, a Global Positioning System (GPS) receiver module 470 may provide additional navigation- and location-related wireless data to the mobile computing device 450, which may be used as appropriate by applications running on the mobile computing device 450.

The mobile computing device 450 may also communicate audibly using an audio codec 460, which may receive spoken information from a user and convert it to usable digital information. The audio codec 460 may likewise generate audible sound for a user, such as through a speaker, e.g., in a handset of the mobile computing device 450. Such sound may include sound from voice telephone calls, may include recorded sound (e.g., voice messages, music files, etc.) and may also include sound generated by applications operating on the mobile computing device 450.

Computing device 400 and/or 450 can also include USB flash drives. The USB flash drives may store operating systems and other applications. The USB flash drives can include input/output components, such as a wireless transmitter or USB connector that may be inserted into a USB port of another computing device.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

Nitrogen Uptake Determined by Laboratory Combustion Analysis Vs. With In-Field Analysis in Corn

Chlorophyll concentration and plant biomass are the basis for determining plant nitrogen content in real-time. In some cases, mass of the primary ear (PEM) of the corn plant may be helpful for determining plant nitrogen status. During reproductive growth stages, nitrogen is remobilized from the stalk and leaves of the plant to the primary ear, making this an important nitrogen sink. When this is the case, plant nitrogen status of the plant is determined using Formula II:

$$Plant Nitrogen Status ( NS ) = α ( PB ) + β ( CC ) + c ( PEM ) .$$

In some cases, diameter of the stalk (SD) of the corn plant may be helpful for determining plant nitrogen status. During early vegetative growth, rate of stalk growth can be limited by nitrogen available for plant uptake. When this is the case, plant nitrogen status of the plant is determined using Formula III:

$$Plant Nitrogen Status ( NS ) = α ( PB ) + β ( CC ) + c ( SD ) .$$

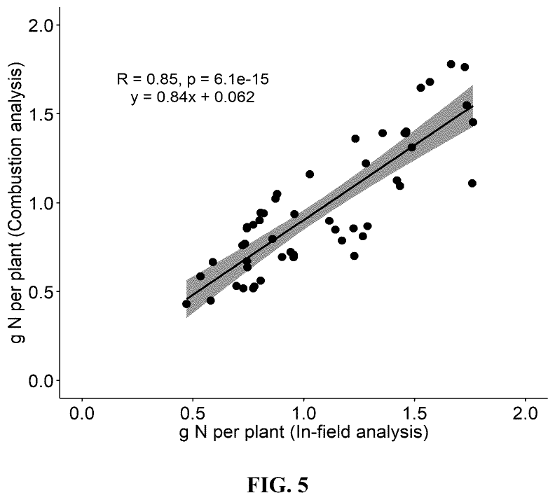

Individual corn plants were sampled during the linear plant nitrogen assimilation growth period. Chlorophyll concentration (CC) was measured on the upper most collared leaf of each plant and the total biomass of the plant (PB) was assessed immediately after sampling. Plant nitrogen uptake (kg N per ha) was calculated as described herein, providing a real-time estimation of plant nitrogen status (NS). Plant nitrogen uptake was also measured for each plant using combustion analysis (see, for example, Miniat et al., supra). Pearson correlation illustrated a significant linear relationship between plant nitrogen uptake measured by the two analyses (FIG. 5).

Use of the Reese-Nevins Assay Infield Trials to Test Plants in the Field with Microbe Vs. Synthetic and/or Synthetic Depletion

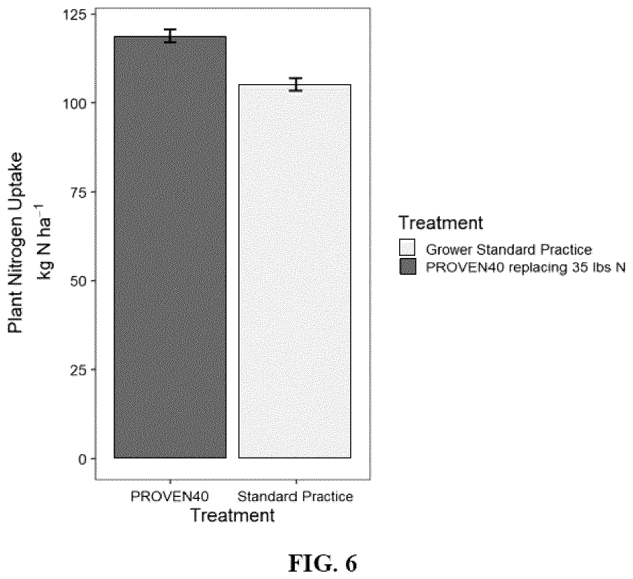

The Reese-Nevins assay, as described herein, was implemented 2325 times (2325 fields sampled) across 34 states. Corn ranged in growth stages from the time the plant had three visible collared leaves (V3) to the kernel dough stage (R4). At 575 fields, 35-40 lbs. of synthetic N was reduced and Pivot Bio PROVEN® 40 was applied. A check strip was left in each field as a comparison area where the full synthetic nitrogen rate was applied (grower standard nitrogen management practice). Plant nitrogen uptake was determined using the assay across these 575 sampling events (FIG. 6).

For example, the assay was completed at a field, when the corn was at the silking growth stage (R1). Six plants were removed from the field in each treatment area (an area in which 35 lbs of synthetic nitrogen was replaced with Pivot Bio PROVEN® 40 and an untreated area with grower standard nitrogen management). Plants were removed by cutting the corn stalk flush with the soil surface and harvesting the aboveground biomass. Each plant was weighed with a hanging scale (AWS-SR-5, American Weigh Scales) and five-gallon bucket. The average biomass of the plants treated with Pivot Bio PROVEN® 40 and 35 lbs less synthetic N was 865 g per plant. The average biomass of the plants in the untreated area with grower standard nitrogen management was 835 g per plant. The chlorophyll concentration of the leaf below and opposite of the primary ear was measured using a chlorophyll meter (MC-100 Chlorophyll Concentration Meter, Apogee Instruments). Chlorophyll concentration was measured in the middle of each leaf at an equal distance between the leaf edge and midrib, and was repeated four times for each leaf. The average chlorophyll concentration of the plants treated with Pivot Bio PROVEN® 40 and 35 lbs less synthetic N was 55.2 μm/m². The average biomass of the plants in the untreated area with grower standard nitrogen management was 48.2 μm/m². Nitrogen status of the plants treated with Pivot Bio PROVEN® 40 and 35 lbs less synthetic N was 703.0 (865*0.80+55.2*0.20=703.0). The nitrogen status of the plants in the untreated area with grower standard nitrogen management was 677.7 (835*0.80+48.2*0.20=677.7). The plants treated with Pivot Bio PROVEN® 40 and 35 lbs less synthetic N had a 3.7% relative increase in nitrogen compared to the plants in the untreated area with grower standard nitrogen management (703.0/677.7−1)*100=3.7%).

Additional variables useful for determining corn plant nitrogen status include mass of the primary ear and diameter of the stalk. Mass of the primary ear may be a useful measurement when determining corn plant nitrogen status during reproductive growth stages from kernel blister (R2) to maturity (R6). Nitrogen begins to accumulate in the corn plant shanks, husks, and cob at late vegetative growth stages (V14-18). At the beginning of reproductive growth stages (R1-2), storage of nitrogen in the shanks, husks, and cobs peaks accounting for approximately 15% of total plant nitrogen. At the same time (R1-2), nitrogen begins to remobilize from the stalk, leaf sheaths, leaf blades, shanks, husks, and cob into the grain. By corn maturity (R6), approximately 70% of total plant nitrogen is in the grain (ear) (Abendroth et al., Corn Growth and Development. Iowa State Univ. Extension publication PMR1009, 2011, available at store.extension. iastate.edu/product/6065; and Bender et al., Agron. J. 105:161-170, 2013). This remobilization and accumulation of nitrogen in the ear from R2-R6 may make the ear an important nitrogen sink to measure late in the corn growing season.

The critical nitrogen uptake phase for corn occurs from the time the plant has five visible collared leaves (V5) to tasseling (VT), and sufficient uptake of N during this period is critical for plant growth and development (Abendroth et al., supra; and Bender et al., supra). Corn stalk diameters can be reduced if plant available nitrogen is limiting to development from V5-VT (Boomsma, et al., Agron. J. 101:1426-1452, 2009), possibly due to mobilization of stem carbohydrate reserves to the ear (Tollenaar et al., “Physiological parameters associated with differences in kernel set among maize hybrids,” pp. 115-130, In M. Westgate and K. Boote (ed.) Physiology and modeling kernel set in maize. Proc. of a Symp. Sponsored by Div. C-2 and A-3 of the CSSA and the ASA, Baltimore, MD. 18-22 Oct. 1998. CSSA and ASA, Madison, WI 2000). Therefore, corn stalk diameter may be an important indicator of plant nitrogen status from V5-VT when paired with leaf chlorophyll concentration and whole plant biomass.

Modifications are made to these methods to assess other crops. For example, the number of plants collected for crops like corn and soybeans for the Reese-Nevins assay is different than for small grains like wheat, barley, or oats. For these small grains, it is difficult to pick six plants for a biomass measurement. At the Feekes 5 growth stage and earlier, one linear foot of row of wheat plants is sampled for a biomass measurement, and a subset of those plants (six leaves) is be used to measure chlorophyll content. Chlorophyll content is measured on the most recent fully developed leaf when sampling before the plant begins heading (before Feekes 10). After the plant begins heading (Feekes 10 and later), the flag leaf is sampled.

Particular embodiments of the invention have been described. Other embodiments are within the scope of the following claims. For example, the steps recited in the claims, described in the specification, or depicted in the figures can be performed in a different order and still achieve desirable results. In some cases, multitasking and parallel processing may be advantageous.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for determining plant nitrogen status of a plant, the method comprising:
(a) determining the chlorophyll content of a plant of a plurality of plants identified in a field or a first region of a field;
(b) determining the biomass of the plant;
(c) normalizing the determined biomass and the determined chlorophyll content for the plant; and
(d) determining plant nitrogen status of the plant using the normalized chlorophyll content (CC) and the normalized biomass (PB) of the plant.

2. (canceled)

3. (canceled)

4. The method of claim 1, wherein the plant nitrogen status of the plant is determined in step (d) using Formula I:
$$Plant Nitrogen Status ( NS ) = α ( PB ) + ϑ ( CC ) .$$

5. The method of claim 4, wherein α is 0.80.

6. The method of claim 4, wherein ϑ is 0.20.

7.-9. (canceled)

10. The method of claim 1, wherein the method further comprises determining an average nitrogen status for the plants of the plurality of plants (NS1).

11. (canceled)

12. (canceled)

13. The method of claim 1, wherein the method further comprises:
(e) determining the chlorophyll content of each plant of an additional plurality of plants identified in a different field or a second region of the field;
(f) determining the biomass of each plant of the additional plurality of plants;
(g) normalizing the determined biomass and the determined chlorophyll content for each plant of the additional plurality of plants;
(h) determining plant nitrogen status of each plant of the additional plurality of plants using the normalized chlorophyll content (CC) and the normalized biomass (PB) for each plant of the additional plurality of plants; and
(i) determining an average nitrogen status for the plants of the additional plurality of plants.

14. (canceled)

15. (canceled)

16. The method of claim 13, wherein the plant nitrogen status of each plant is determined in step (h) using Formula I:
$$Plant Nitrogen Status ( NS ) = α ( PB ) + ϑ ( CC ) .$$

17. The method of claim 16, wherein α is 0.80.

18. The method of claim 16, wherein ϑ is 0.20.

19. (canceled)

20. The method of claim 13, wherein the method further comprises determining the relative nitrogen status of the plurality of plants as compared to the additional plurality of plants comprising comparing the NS1 to the average nitrogen status of the plants of the additional plurality of plants (NS2).

21. The method of claim 20, wherein the relative nitrogen status is determined using Formula II:
$$Relative Nitrogen Status = ( NS 1 / NS 2 ) - 1.$$

22. The method of claim 21, wherein the method further comprises determining a nitrogen performance index (NPI) using Formula III:
$$NPI = ( Relative Nitrogen Status ) \times 100 \% .$$

23. (canceled)

24. The method of claim 13, wherein the additional plurality of plants comprises at least six plants.

25.-27. (canceled)

28. The method of claim 20, wherein the method further comprises determining plant nitrogen content per acre using the relative nitrogen status and the uptake of nitrogen by growth stage.

29.-35. (canceled)

36. The method of claim 13, wherein the plants of the plurality of plants are provided a different nitrogen treatment than the plants of the additional plurality of plants.

37. The method of claim 36, wherein the different nitrogen treatments comprise treatment with synthetic nitrogen and treatment with nitrogen fixing microbes.

38. The method of claim 36, wherein the different nitrogen treatments comprise treatment with different nitrogen fixing microbes.

39. The method of claim 37, wherein the nitrogen fixing microbes comprise microbes identified in Table 1.

40. (canceled)

41. One or more machine-readable storage devices storing instructions that are executable by one or more processing devices, and upon such execution cause the one or more processing devices to perform operations comprising:
(a) receiving a first input on a chlorophyll content of a plant of a plurality of plants identified in a field or a first region of a field;
(b) receiving a second input on a biomass of the plant;
(c) normalizing the biomass and the chlorophyll content for the plant;
(d) determining plant nitrogen status of the plant using the normalized chlorophyll content (CC) and the normalized biomass (PB) of the plant; and
(e) outputting the determined plant nitrogen status.

42. The one or more machine-readable storage device of claim 41, wherein the plant nitrogen status of the plant is determined in step (d) using Formula I:
$$Plant Nitrogen Status ( NS ) = α ( PB ) + ϑ ( CC ) .$$

43. The one or more machine-readable storage devices of claim 42, wherein α is 0.80.

44. The one or more machine-readable storage devices of claim 42, wherein ϑ is 0.20.

45.-47. (canceled)

48. The one or more machine-readable storage devices of claim 41, wherein the operations further comprise determining an average nitrogen status for the plants of the plurality of plants (NS1).

49. The one or more machine-readable storage devices of claim 41, wherein the operations further comprise:
(e) determining the chlorophyll content of each plant of an additional plurality of plants identified in a different field or a second region of the field;
(f) determining the biomass of each plant of the additional plurality of plants;
(g) normalizing the determined biomass and the determined chlorophyll content for each plant of the additional plurality of plants;
(h) determining plant nitrogen status of each plant of the additional plurality of plants using the normalized chlorophyll content (CC) and the normalized biomass (PB) for each plant of the additional plurality of plants; and
(i) determining an average nitrogen status for the plants of the additional plurality of plants.

50. The one or more machine-readable storage devices of claim 49, wherein the plant nitrogen status of each plant is determined in step (h) using Formula I:
$$Plant Nitrogen Status ( NS ) = α ( PB ) + ϑ ( CC ) .$$

51. The one or more machine-readable storage devices of claim 50, wherein α is 0.80.

52. The one or more machine-readable storage devices of claim 51, wherein ϑ is 0.20.

53. (canceled)

54. The one or more machine-readable storage devices of claim 49, wherein the operations further comprise determining the relative nitrogen status of the plurality of plants as compared to the additional plurality of plants comprising comparing the NS1 to the average nitrogen status of the plants of the additional plurality of plants (NS2) using Formula II:
$$Relative Nitrogen Status = ( NS 1 / NS 2 ) - 1.$$

55. (canceled)

56. The one or more machine-readable storage devices of claim 54, wherein the operations further comprise determining a nitrogen performance index (NPI) using Formula III:
$$NPI = ( Relative Nitrogen Status ) \times 100 \% .$$

57.-105. (canceled)
